(19) United States
(12) Patent Application Publication
(10) Pub. No.: US 2025/0155420 A1
(43) Pub. Date: May 15, 2025

(54) SYSTEM FOR PROVIDING SAFE WATER QUALITY INFORMATION OF DRINKING WATER AND METHOD OF SUPPLYING SAFE POTABLE WATER USING THE SAME

(71) Applicant: Bluesen Co., Ltd., Daegu (KR)

(72) Inventors: Changsik SON, Busan (KR); Youjung KIM, Busan (KR); Kyungyup HWANG, Busan (KR); Hyunchul CHOI, Busan (KR); Kyungsuk YU, Daegu (KR); Jiun LEE, Daegu (KR); Kyungwoo KIM, Busan (KR)

(21) Appl. No.: 18/674,170

(22) Filed: May 24, 2024

(30) Foreign Application Priority Data
Nov. 9, 2023 (KR) 10-2023-0154578

(51) Int. Cl. G01N 33/18

(52) U.S. Cl. CPC G01N 33/18

(57) ABSTRACT

An embodiment relates to a system for providing safe water quality information of drinking water capable of supplying safe drinking water and a method of supplying safe potable water using the same, and more specifically, to a system for providing safe water quality information of drinking water configured to measure water quality state of tap water to be supplied in real time, calculate a disinfection ability and a food poisoning index reflecting the measured water quality state and weather information, and calculate a carbon emission reduction amount, which is environmental improvement information resulting from using tap water as drinking water and display the same along with the water quality state.

### CROSS-REFERENCE TO RELATED APPLICATION

This application claims the benefit of Korean Patent Application No. 10-2023-0154578 filed on Nov. 9, 2023, in the Korean Intellectual Property Office, the entire disclosure of which is incorporated herein by reference for all purposes.

### BACKGROUND

### 1. Field of the Invention

The present disclosure relates to a system for providing safe water quality information of drinking water capable of supplying safe drinking water (hereinafter, used interchangeably with “potable water”) and a method of supplying safe potable water using the same, and more specifically, to a system for providing safe water quality information of drinking water that provides safe potable water that people can drink with confidence, by measuring water quality state of tap water to be supplied in real time, calculating a disinfection ability and a food poisoning index reflecting the measured water quality state and weather information, and calculating a carbon emission reduction amount, which is environmental improvement information resulting from using tap water as drinking water and displaying the same along with the water quality state, sharing the water quality state between potable water information providing units, and when the supplied tap water is unfit for drinking, guiding a tap water supply location managed by the potable water information providing unit that provides drinkable tap water of good water quality, and a method of supplying safe potable water using the same.

### 2. Description of the Related Art

Every year, the government spends trillions of dollars in budget and effort to produce clean and safe tap water.

However, due to distrust in the safety of tap water, people do not drink tap water directly, but instead pay high costs to use water purifiers or purchase bottled water to drink.

Although tap water is cleanly treated and supplied, most tap water is used for non-drinking purposes such as washing dishes, laundry, and bathing.

Therefore, the government is spending a lot of effort and high costs to increase the drinking rate of tap water. Although these efforts have increased people's satisfaction with and use of tap water, the drinking rate of using tap water for drinking remains at around 5%.

In addition, even when using a water purifier, the number of damage and complaint cases reported to the Korea Consumer Protection Agency due to water quality problems of the water purifier due to poor water purifier management is increasing every year.

It has also been revealed that one out of three bottled water sellers have been selling drinking spring water supplied by a company that violates water quality standards.

In order to solve these problems, Korean Patent No. 10-1632732 (published on Jun. 23, 2016), titled Dispenser Water Quality Information Providing System (hereinafter referred to as “prior art”), provides reliability for tap water supplied to a same water quality facility zone by measuring water quality of a main dispenser installed in the same water quality facility zone, providing the measured water quality to other dispensers in the same water quality facility zone to display the same to make the public aware of the safety of tap water.

Although the above prior art provides a certain level of reliability to the public, there are limitations in providing the public with reliability regarding tap water by displaying only simple numbers.

This is because the public still has vague anxiety about the water quality of tap water, and measures to fundamentally resolve this are required.

### SUMMARY

Therefore, an object of the present disclosure is to provide a system for providing safe water quality information of drinking water that provides safe potable water that people can drink with confidence, by measuring water quality state of tap water to be supplied in real time, calculating a disinfection ability and a food poisoning index reflecting the measured water quality and weather information, and calculating a carbon emission reduction amount, which is environmental improvement information resulting from using tap water as drinking water and displaying the same along with the water quality state, sharing the water quality state between potable water information providing units, and when the supplied tap water is unfit for drinking, guiding a tap water supply location managed by the potable water information providing unit that provides drinkable tap water of good water quality, and a method of supplying safe potable water.

A system for providing safe water quality information of drinking water according to the present disclosure to achieve the above object includes: a plurality of potable water information providing units installed in different water supply zones, each configured to receive tap water from a water pipe of a water supply zone of the same water quality, measure water quality of the tap water, if the measured water quality exceeds a standard value, perform a discharge setting that commands to discharge the supplied tap water, and if the measured water quality is below the standard value, perform a permission setting that permits to supply as potable water which is drinking water, display and then transmit water quality measurement information including water quality state information according to the measured water quality and own location information, display environmental improvement information due to use of the tap water as drinking water and potable water reliability information based on the water quality state information, and display received potable water living information; and a potable water living information providing unit configured to receive the water quality measurement information from the potable water information providing unit, analyze water quality state based on the location information of the water quality measurement information to generate regional potable water information, and generate the potable water living information including the regional potable water information to transmit it to the potable water information providing unit.

The potable water information providing unit may include: a potable water information terminal installed at a random location of the water supply zone and configured to receive tap water from the water pipe of the water supply zone and measure water quality of the tap water, display and then transmit the water quality measurement information including the water quality state information for the measured water quality, the environmental improvement information, and the potable water reliability information based on the water quality state information, receive, display, and then transmit the potable water living information, if the water quality exceeds the standard value, perform the discharge setting to transmit a tap water discharge command, and if the water quality is below the standard value, perform the permission setting to permit the tap water as potable water which is drinking water, and transmit a potable water release permission command; and a smart dispenser installed at a random location of the water supply zone and configured to receive tap water from the water pipe of the water supply zone, discharge the supplied tap water to outside when the tap water discharge command is received from the potable water information terminal, set a potable water release permission when the potable water release permission command is received and release the tap water upon tap water release request while the potable water release permission is set, and receive and display the water quality state information, the environmental improvement information, the potable water reliability information, and the potable water living information from the potable water information terminal.

The potable water information terminal may include: a storage unit configured to store own location information; a communication unit configured to perform data communication with the potable water living information providing unit and the smart dispenser; a display unit; a water quality measuring unit configured to receive the tap water to measure and output the water quality of the tap water; and a controller configured to measure the water quality of the tap water through the water quality measuring unit, display and then transmit the water quality measurement information including the water quality state information for the measured water quality, the environmental improvement information, and the potable water reliability information based on the water quality state information, receive, display and then transmit the potable water living information, if the water quality exceeds the standard value, perform the discharge setting and transmit the tap water discharge command to the smart dispenser through the communication unit, and if the water quality is below the standard value, perform the permission setting to permit the tap water as drinking water for drinking and transmit the potable water release permission command to the smart dispenser through the communication unit, and further include the number of discharges, a discharge amount, the number of releases, and a release amount of tap water according to discharge and release of tap water through the smart dispenser in the water quality measurement information and transmit the water quality measurement information to a potable water living information management unit through the communication unit.

The potable water information terminal may further include an input unit configured to, together with the display unit, provide a user interface means through which a potable water information terminal user inputs and selects information for using the potable water information terminal, and the controller may include: a terminal registration unit configured to acquire the location information of the potable water information terminal and store the location information in the storage unit, generate dispenser registration information including the location information and transmit the dispenser registration information to the potable water living information providing unit through the communication unit to register the potable water information terminal and a location of the potable water information terminal; a water quality information acquisition unit configured to, when a water quality measuring event occurs, acquire water quality information of the tap water through the water quality measuring unit, and generate and output the water quality state information including the acquired water quality information; a water quality management controller configured to acquire the water quality state information from the water quality information acquisition unit, determine whether the water quality of the water quality state information is below a preset standard value to determine whether to discharge tap water and whether to permit release, and when it is determined to discharge tap water, perform the discharge setting and transmit the tap water discharge command to the smart dispenser, and when it is determined to permit release, set the potable water release permission and transmit the potable water release permission command to the smart dispenser; a carbon emission reduction amount calculating unit configured to define a reduced carbon amount compared to using plastic bottled water according to a standard release amount (drinking amount) of tap water or a reduced carbon amount compared to using a water purifier according to the standard release amount of tap water, and calculate and store a carbon emission reduction amount which is the environmental improvement information, in correspondence to the defined carbon amount and the release amount of drinking tap water; a water quality information providing unit configured to acquire the water quality state information and the carbon emission reduction amount, display the water quality measurement information including the water quality state information and the carbon emission reduction amount on the display unit, and transmit them to the potable water living information providing unit and the smart dispenser through the communication unit; and an information processing unit configured to receive the potable water living information from the potable water living information providing unit through the communication unit and display it on the display unit.

The potable water information terminal may further include a location measuring unit configured to receive location data from a plurality of Global Positioning System (GPS) satellites to measure own location, and output measured location information to the controller, and the terminal registration unit of the controller may be configured to acquire the location information through the location measuring unit.

The potable water information terminal and the smart dispenser may further include an approach detection sensor unit configured to detect users approaching within a predetermined distance and output a detection signal, and each of the controller of the potable water information terminal and a dispenser controller of the smart dispenser may further include a promotional video output unit configured to, when the detection signal is input from the corresponding approach detection sensor unit, acquire a promotional video promoting features and effects of the potable water information providing unit and display the promotional video through the display unit of the potable water information terminal, and transmit and display the promotional video to a dispenser display unit of the smart dispenser.

The potable water living information may include GIS-based potable water information in which water quality state information for each of the potable water information terminal and nearby potable water information terminals is mapped to GIS information, and the information processing unit may be configured to display the GIS-based potable water information on the display unit, and display detailed water quality state information for the potable water information terminal selected through the input unit on the display unit.

The water quality information providing unit may be configured to count the number of potable water releases through a potable water release controller and the smart dispenser and the number of tap water discharges through the water quality management controller and the smart dispenser, and include the number of potable water releases, the release amount according to the number of potable water releases, the number of tap water discharges, and the discharge amount according to the number of discharges in the water quality measurement information and transmit the water quality measurement information to the potable water living information providing unit, and the information processing unit may be configured to extract, from the potable water living information including statistics information generated by the potable water living information providing unit by combining with GIS after generating statistics based on the water quality state information of the water quality measurement information, the number of potable water discharges and discharge amount, and the number of potable water releases and release amount received from the potable water information terminal, the statistics information and display the statistics information on the display unit.

The potable water living information providing unit may include: a server storage unit including a water quality information DB storing water quality state information over time for each of potable water information terminals; a server communication unit configured to perform mutual data communication with the potable water information terminals; and a server controller configured to receive the water quality measurement information from the potable water information terminal through the server communication unit, store the water quality measurement information in the server storage unit, analyze the water quality state based on the location information of the water quality measurement information to generate the regional potable water information, and generate the potable water living information including the regional potable water information to transmit it to the potable water information terminal.

The server storage unit may further include a dispenser DB storing dispenser registration information including potable water information terminal installation location information for each potable water information terminal, and the server controller may include: a terminal management unit configured to, when the dispenser registration information is received from any potable water information terminal through the server communication unit, store and register the dispenser registration information in the dispenser DB; a water quality information collecting unit configured to collect and output the water quality measurement information from the potable water information terminal registered in the dispenser DB through the server communication unit; a water quality statistics information generating unit configured to, after generating statistics information for the water quality state information of the water quality measurement information for each potable water information terminal, the number of discharges and a discharge amount of tap water, and the number of releases and a release amount of potable water collected through the water quality information collecting unit, generate and output GIS-based statistics information in which GIS is combined with the water quality state information and the statistics information in correspondence to the location of the potable water information terminal of the water quality measurement information; and a regional information providing unit configured to generate regional water quality state information according to the location of the potable water information terminal based on the water quality state information of the water quality measurement information acquired through the water quality information collecting unit, and transmit one or more of the regional water quality state information and the GIS-based statistics information output from the water quality statistics information generating unit to the potable water information terminal corresponding to the region through the server communication unit.

The server controller may further include a dispenser abnormality determining unit configured to, when water quality of any potable water information terminal falls below a predetermined standard value compared to water quality of other adjacent potable water information terminals based on the water quality measurement information collected through the water quality information collecting unit, determine that there is an abnormality in the potable water information terminal of poor water quality or that there is an abnormality in the water pipe supplying tap water to the potable water information terminal, and transmit potable water abnormality notification information to a preset manager terminal.

The server controller may further include a weather information collecting unit configured to collect and output weather information for each water supply zone from any one of a weather system and the potable water information terminal of the potable water information providing unit for the water supply zone through the server communication unit, and the regional information providing unit may be configured to, based on the weather information for each water supply zone and the water quality state information for each water supply zone, calculate in real time a disinfection ability and a food poisoning index for each water supply zone, which are the potable water reliability information, and further provide them to the potable water information terminal of the corresponding potable water information providing unit for each water supply zone.

A method of supplying safe potable water using a system for providing safe water quality information of drinking water according to the present disclosure to achieve the above object includes: a potable water information providing unit registrating process in which a potable water information providing unit installed in a water supply zone where tap water of the same water quality is supplied acquires own location information and stores it in a storage unit, and generates dispenser registration information including the location information and transmits it to a potable water living information providing unit to register own location; a water quality measurement information providing process in which the potable water information providing unit receives tap water from a water pipe of the water supply zone, measures water quality of the tap water, if the measured water quality exceeds a standard value, performs a discharge setting, and if the measured water quality is below the standard value, performs a permission setting to permit as potable water which is drinking water, and transmits water quality measurement information including water quality state information according to the measured water quality and own location information; a potable water living information providing process in which the potable water living information providing unit receives the water quality measurement information from the potable water information providing unit, analyzes water quality state based on the location information of the water quality measurement information to generate regional potable water information including regional water quality state information, and generates potable water living information including the regional potable water information to transmit the potable water living information to the potable water information providing unit; and a potable water information display process in which the potable water information providing unit receives and display the received potable water living information, and generates and displays environmental improvement information due to use of tap water as drinking water based on the water quality measurement information and the potable water living information and potable water reliability information based on the water quality state information.

The water quality measurement information providing process may include: a water quality management controlling step in which a potable water information terminal of the potable water information providing unit receives tap water from the water pipe of the water supply zone and measures water quality of the tap water, determine whether the water quality exceeds or is below the standard value, if the water quality exceeds the standard value, performs the discharge setting by determining to discharge the supplied tap water and notifies the discharge setting of the tap water by transmitting a tap water discharge command to one or more smart dispensers of the water supply zone, and if the water quality is below the standard value, performs the permission setting to permit the tap water as potable water which is drinking water, and sets a drinking permission by transmitting a potable water release permission command to the smart dispenser; and a tap water treating step in which each smart dispenser of the potable water information providing unit discharges the supplied tap water upon the discharge setting of the tap water, and when a release request of the tap water occurs upon the drinking permission setting of the tap water, releases the tap water as drinking water.

The potable water information display process may include: a water quality information providing step in which a controller of the potable water information terminal acquires the water quality state information from a water quality information acquisition unit through a water quality information providing unit, displays water quality measurement information including the water quality state information on a display unit, and transmits the water quality measurement information to the potable water living information providing unit and the smart dispenser through a communication unit; and an information processing step in which the controller displays the environmental improvement information due to use of tap water as drinking water and the potable water reliability information based on the water quality state information through the water quality information acquisition unit, receives the potable water living information from the potable water living information providing unit through the information processing unit and displays it on the display unit.

The potable water information display process may further include a location information acquiring step in which the controller receives location data from a plurality of Global Positioning System (GPS) satellites through a location measuring unit and measures own location, and outputs measured location information to the controller, and the controller may acquire, in a dispenser registration step, the location information through the location measuring unit.

The potable water information display process may further include: a user approach detecting step in which the controller detects users approaching within a predetermined distance through an approach detection sensor unit and outputs a detection signal; and a promotional video output step in which, when the detection signal is input from the approach detection sensor unit, the controller acquires a promotional video promoting features and effects of the potable water information terminal through a promotional video output unit and displays the promotional video on the display unit.

The potable water living information may include GIS-based potable water information in which water quality state information for each of the potable water information terminal and nearby potable water information terminals is mapped to GIS information, and in the information processing step, the information processing unit may display the GIS-based potable water information on the display unit, and display detailed water quality state information for the potable water information terminal selected through an input unit on the display unit.

In the water quality information providing step, the controller may count the number of potable water releases through a potable water release controller and the number of tap water discharges through a water quality management controller, and include the number of potable water releases, a release amount according to the number of potable water releases, the number of tap water discharges, and a discharge amount according to the number of tap water discharges in the water quality measurement information and transmits the water quality measurement information to the potable water living information providing unit, and in the information processing step, the controller may extract, from the potable water living information including statistics information generated by the potable water living information providing unit by combining with GIS after generating statistics based on the water quality state information of the water quality measurement information, the number of potable water discharges and discharge amount, the number of potable water releases and release amount received from the potable water information terminal, the statistics information and displays the statistics information on the display unit.

The potable water living information providing process may include: a water quality information collecting step in which a server controller of the potable water living information providing unit collects the water quality measurement information from a potable water information terminal registered in a dispenser DB through a water quality information collecting unit; a water quality statistics information generating step in which, after generating statistics information for the water quality state information of the water quality measurement information for each potable water information terminal, the number of discharges and a discharge amount of tap water, and the number of releases and a release amount of potable water collected from the water quality information collecting unit, the server controller generates and outputs GIS-based statistics information in which GIS is combined with the water quality state information and the statistics information in correspondence to the location of the potable water information terminal of the water quality measurement information through a water quality statistics information generating unit; and a regional information providing step in which the server controller generates regional water quality state information according to the location of the potable water information terminal based on the water quality state information of the water quality measurement information input from the water quality information collecting unit, and transmits one or more of the regional water quality state information and the GIS-based statistics information output from the water quality statistics information generating unit to the potable water information terminal corresponding to the region through the server communication unit through a regional information providing unit.

The potable water living information providing process may further include a weather information collecting step in which the server controller collects and outputs weather information for each water supply zone from any one of a weather system and the potable water information providing unit through a weather information collecting unit, and in the regional information providing step, the server controller may calculate in real time, based on the weather information for each water supply zone and the water quality state information for each water supply zone, a disinfection ability and a food poisoning index for each water supply zone, which are the potable water reliability information, and further provide them to the potable water information terminal of the corresponding potable water information providing unit for each water supply zone through the regional information providing unit.

The potable water living information providing process may further include a dispenser abnormality determining step in which, when water quality of any potable water information terminal falls below a predetermined standard value compared to water quality of adjacent potable water information terminals based on the water quality measurement information collected from the water quality information collecting unit, the server controller determines that there is an abnormality in the potable water information terminal of poor water quality or that there is an abnormality in a water pipe supplying tap water to the potable water information terminal, and transmits potable water abnormality notification information to a preset manager terminal through a dispenser abnormality determining unit.

The present disclosure has an effect of increasing the reliability of tap water by collecting water quality state information including water quality information based on the location of the potable water information providing unit installed in a water supply zone composed of water pipes supplying tap water of the same water quality, generating water quality statistics information for each water supply zone where a potable water information terminal from which the water quality state information is collected is installed and then displaying on the potable water information terminal of the potable water information providing unit.

In addition, the present disclosure has effects of intuitively delivering environmental improvement information by checking the release amount when drinking tap water in addition to environmental improvement information including the carbon emission reduction amount by drinking tap water upon detecting a person approaching the potable water information terminal or the smart dispenser of the potable water information providing unit, and graphically displaying the results of automatically calculating the amount of carbon dioxide saved compared to purified or bottled water, and reducing vague anxiety about tap water and at the same time encouraging people to use tap water as drinking water by giving them a sense of pride in contributing to environmental improvement by displaying a promotional video containing information about the cleanliness of tap water of the potable water information terminal on the potable water information providing unit.

In addition, the present disclosure has an effect of quickly handling failures of the potable water information providing unit due to abnormalities in the potable water information providing unit and pipe abnormalities in the water supply zone, or quickly performing tap water pipe construction in the water supply zone, by analyzing the water quality measurement information for each potable water information providing unit and an automatic discharge amount according to water quality abnormalities, and requesting a check of failure of the potable water information providing unit with an automatic discharge amount exceeding a standard value or a pipe inspection in a specific zone where the potable water information providing unit is installed.

In addition, by calculating the disinfection ability and food poisoning index based on weather information including temperature, humidity, etc., based on the location of the potable water information providing unit and measured water quality state information and providing them for citizens to view, the present disclosure has an effect of encouraging drinking so that citizens of the corresponding region can drink tap water with safety.

### BRIEF DESCRIPTION OF THE DRAWINGS

FIG. 1 is a diagram illustrating a configuration of a system for providing safe water quality information of drinking water according to the present disclosure.

FIG. 2 is a diagram illustrating a configuration of a potable water information terminal of a potable water information providing unit of the system for providing safe water quality information of drinking water according to the present disclosure.

FIG. 3 is a diagram illustrating a configuration of a smart dispenser of the potable water information providing unit of the system for providing safe water quality information of drinking water according to the present disclosure.

FIG. 4 is a diagram illustrating a configuration of a potable water living information providing unit of the system for providing safe water quality information of drinking water according to the present disclosure.

FIG. 5 is a flowchart illustrating a method of supplying safe potable water using the system for providing safe water quality information of drinking water according to an embodiment of the present disclosure.

FIG. 6 is a flowchart illustrating a promotional video providing method of the potable water information terminal of the method of supplying safe potable water using the system for providing safe water quality information of drinking water according to an embodiment of the present disclosure.

### DETAILED DESCRIPTION

Hereinafter, with reference to the accompanied drawings, configurations and operations of a system for providing safe water quality information of drinking water according to the present disclosure will be described in detail, and a method of supplying safe potable water using the system will be described.

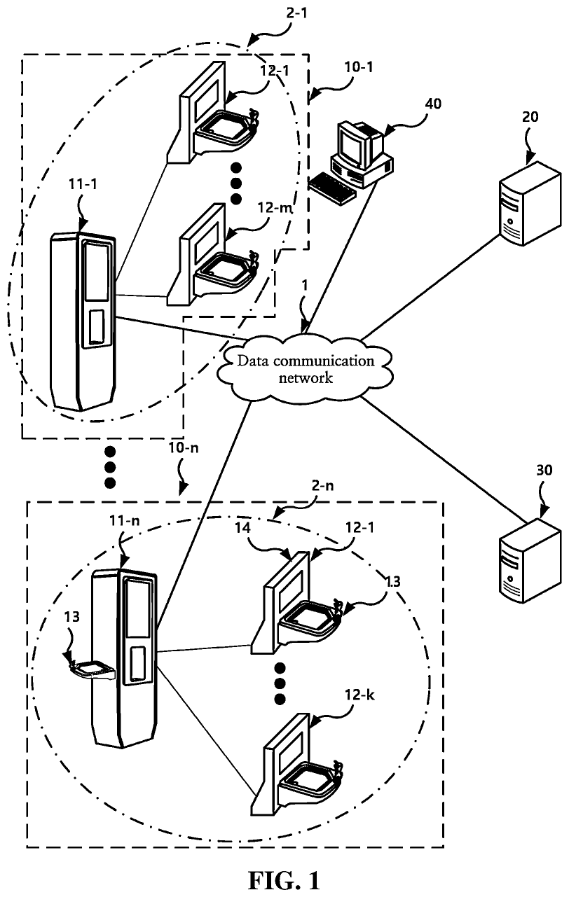

FIG. 1 is a diagram illustrating a configuration of a system for providing safe water quality information of drinking water according to the present disclosure.

Referring to FIG. 1, the system for providing safe water quality information of drinking water according to the present disclosure includes a plurality of potable water information providing units 10 and a potable water living information providing unit 20, and may further include a weather system 30 and one or more manager terminal units 40 according to embodiments.

The potable water information providing unit 10, the potable water living information providing unit 20, the weather system 30, and the manager terminal unit 40 are connected through a data communication network 1 to perform mutual data communication.

The data communication network 1 may be a communication network in which one or more of a local area network (LAN) including a WiFi network, an Internet network including a wide area network (WAN), a mobile communication network such as 3rd generation (3G), 4G, and 5G are combined.

The potable water information providing unit 10 is installed in a water supply zone 2 where a water pipe supplying tap water of the same quality is installed, and is installed in the water supply zone 2 while including one or more potable water information terminals 11 and optionally including one or more smart dispensers 12. The potable water information terminal 11 may include a dispenser to release tap water as drinking water, or may display only information indicating that the tap water is potable water of good water quality for use as drinking water according to the present disclosure without a dispenser.

The potable water information providing unit 10 has location information (which may include location information about the water supply zone 2) corresponding to the location (and/or water supply zone location) where it (potable water information terminal 11) is installed, and is connected to a water pipe passing through the water supply zone 2 to receive tap water, measures the water quality of the supplied tap water, displays the water quality state on the potable water information terminal 11 and the smart dispenser 12, and releases tap water as drinking water upon potable water release request from any one or more of the potable water information terminal 11 and the smart dispenser 12. The location information may include one or more of longitude and latitude and unique identification information of the potable water information terminal 11, and may further include a region name.

The potable water information terminal 11 is configured to, when including a dispenser 13 according to the embodiment, if the measured tap water quality exceeds a preset standard value, perform a discharge setting, and then discharge all the tap water it has received to the outside, i.e., into sewage, count the number of discharges, calculate a total discharge amount by multiplying a discharge amount per minute by a discharge time, and store the total discharge amount. In addition, if the measured tap water quality is below the standard value, the potable water information terminal 11 performs a potable water release permission setting, and then releases tap water as drinking water upon potable water release request, counts the number of releases, calculates a total release amount by multiplying a release amount per minute by a release time, and stores the total release amount. Further, the potable water information terminal 11 transmits the tap water discharge command to the smart dispenser 12 after the discharge setting, and transmits the potable water release permission command to the smart dispenser 12 after the potable water release permission setting.

On the other hand, if the dispenser 13 is not provided, the potable water information terminal 11 will perform a discharge setting upon discharge determination and transmit a tap water discharge command to the smart dispenser 12, and will perform a potable water release permission setting upon permission determination and transmit a potable water release permission command to the smart dispenser 12.

The potable water information terminal 11 generates water quality state information including the one or more measured water quality information, generates water quality measurement information including one or more of the water quality state information, the location information, the number of tap water discharges and discharge amount, the number of potable water releases and the potable water release amount and transmits to the potable water living information providing unit 20 through the data communication network 1. The water quality information may include information on one or more of turbidity, chlorine concentration, hydrogen ion concentration, and water temperature.

The potable water information terminal 11 may use one or more of the measured water quality information and weather information such as temperature and humidity to calculate a disinfection ability and a food poisoning index, which is potable water reliability information that improves the reliability of tap water, and may also display and transmit the water quality measurement information by further including the disinfection ability and food poisoning index.

In addition, the potable water information terminal 11 transmits and displays the water quality state information, disinfection ability, and food poisoning index to the smart dispenser 12 upon update of the water quality state information, disinfection ability, and food poisoning index or upon request from the smart dispenser 12.

The potable water information terminal 11 receives potable water living information from the potable water living information providing unit 20 and displays on own display means or transmits to the smart dispenser 12 and displays on the display means of the smart dispenser 12. The potable water living information may include ultraviolet ray index, today's weather, etc., and potable water information including the water quality state information for each water supply zone, disinfection ability, food poisoning index, carbon emission reduction, etc., may be included.

The smart dispenser 12 may be connected to the potable water information terminal 11 through the data communication network 1, and is directly connected to the potable water information terminal 11 through a communication method such as RS-485, universal serial bus (USB), or Wi-Fi Direct to perform data communication, receives information such as the water quality state information, disinfection ability, and food poisoning index from the potable water information terminal 11 and displays on the display means.

The smart dispenser 12 includes an information receiving unit 14 including a communication function, display means, water treatment means, control means, etc., and a drinking fountain 13 connected to the water pipe of the same water supply zone 2 as the potable water information terminal 11, receives tap water, and releases tap water under the control of the information receiving unit 14.

The smart dispenser 12 calculates the number of discharges and discharge amount of tap water discharged, calculates the number of releases and releases amount, and then provides drinking water treatment information, including the number of discharges, discharge amount, number of releases, release amount, etc., to the potable water information terminal 11.

Then, in the case that the potable water information terminal 11 includes its own drinking fountain 13, it calculates the total release amount by adding the release amount input from the smart dispenser 12 to the release amount of tap water released by itself, and calculates and stores the total discharge amount by adding the discharge amount input from the smart dispenser 12 to the discharge amount of the tap water discharge by itself. The calculated information is provided to the smart dispenser 12 and the potable water living information providing unit 20.

Meanwhile, in the case that the potable water information terminal 11 includes its own drinking fountain 13 according to the embodiment, it generates environmental improvement information by calculating the carbon emission reduction amount for the total release amount by adding the release amount of tap water released by itself and the release amount input from the smart dispenser 12, and provides the generated environmental improvement information to the smart dispenser 12 and the potable water living information providing unit 20.

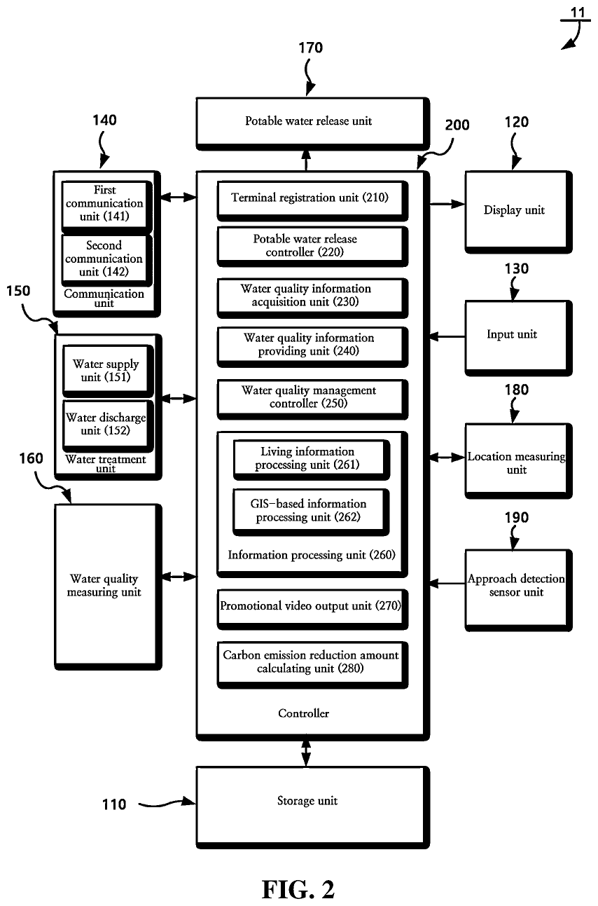

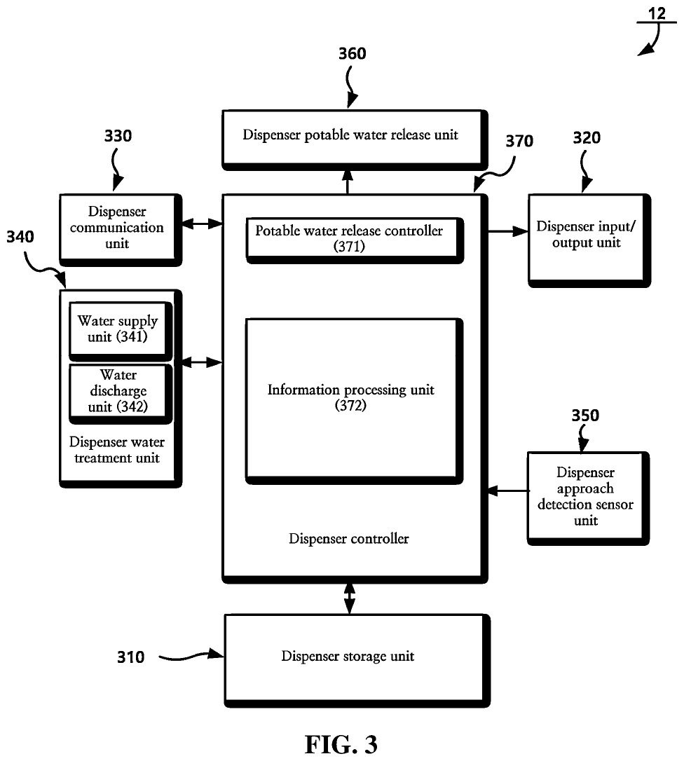

The detailed configuration of the potable water information terminal 11 and the smart dispenser 12 will be described in detail with reference to FIGS. 2 and 3 below.

The potable water living information providing unit 20 collects water quality measurement information from a plurality of potable water information terminals 11, and generates potable water living information for each potable water information terminal 11 based on the collected water quality measurement information and transmits it to each corresponding potable water information terminal 11.

According to another embodiment, the potable water living information providing unit 20 may collect information such as tap water discharge amount and tap water release amount, excluding direct water quality measurement information from the smart dispenser 12 as well as the plurality of potable water information terminals 11. In this case, the smart dispenser 12 must be registered in the potable water living information providing unit 20.

The potable water living information includes water quality information for each water supply zone 2 where the potable water information terminal 11 and potable water information terminals 11 in the vicinity of the potable water information terminal 11 or across the country are installed, or GIS-based water quality state information combined with water quality information and map data (geographic information system (GIS)). In addition, the potable water living information may further include weather information including weather, air quality, etc., corresponding to the water supply zone 2 at the location where the potable water information terminal 11 is installed.

In addition, in according to another embodiment, the potable water living information providing unit 20 may be configured to calculate the food poisoning index for each location based on weather information such as humidity and temperature at each location where the potable water information terminal 11 is installed and water quality state information of the water quality measurement information to display on the potable water information terminal 11 and the smart dispenser 12 of the potable water information providing unit 10.

In addition, the potable water living information providing unit 20 may calculate, display and store the disinfection ability based on the residual chlorine, PH, and water temperature included in the water quality state information of the water quality measurement information received from the water quality information providing unit 10, and then provide and display to the potable water information providing unit 10.

In addition, the potable water living information providing unit 20 may transmit statistics information on water quality measurement information provided from the potable water information terminals 11 as included in the potable water living information. Statistics information may be transmitted by further including information such as daily, weekly, and annual number of tap water discharges, tap water discharge amount, number of potable water releases, potable water release amount, and carbon emission reduction amount according to the potable water release amount for each potable water information terminal 11, in the potable water living information. In addition, the potable water living information may include various regional notices and information for convenience.

The weather system 30 provides weather information including regional weather, air quality, UV index, and the like.

The manager terminal unit 40 is a terminal used by managers of regional government offices, regional public institutions, etc., and managers of potable water information terminals, which receives local notices and benefit information from the manager and provides them to the potable water living information providing unit 20 for registration.

FIG. 2 is a diagram illustrating a configuration of a potable water information terminal of the system for providing safe water quality information of drinking water according to the present disclosure.

Referring to FIG. 2, the potable water information terminal 11 includes a storage unit 110, a display unit 120, an input unit 130, a communication unit 140, a water quality measuring unit 150, and a controller 200, and according to embodiments, further includes a drinking fountain 13, a water treatment unit 160, a potable water release unit 170, a location measuring unit 180, and an approach detection sensor unit 190.

The storage unit 110 includes a program area for storing a control program for controlling the overall operation of the potable water information terminal 11 according to the present disclosure, a temporary area for temporarily storing data generated during execution of the control program, and a data area for semi-permanently storing data required for performing the control program and data generated during execution of the control program. In the data area, water quality state information including measured water quality information, location information, counted number of tap water discharges, tap water discharge amount, number of potable water releases, potable water release amount, disinfection ability, food poisoning index, received potable water living information, etc, may be stored.

The display unit 120 may be a liquid crystal display (LCD) or an organic EL display device, and displays various information in one or more of text, graphics, video, and still images.

The input unit 130 includes one or more of a button input device including a potable water discharge button, and a water selection button for hot water, cold water, lukewarm water, etc., and a touch pad that is integrated with the screen of the display unit 120 and outputs a location signal for the touched location to enable performing button selection, information input, selectable location selection by touching the potable water information terminal 11 close to own current location in the map data displayed on the screen displayed on the display unit 120, etc, through a variety of user interfaces including soft buttons corresponding to the potable water discharge button, water selection button for hot water, cold water, lukewarm water, etc., displayed on the screen of the display unit 120.

The communication unit 140 includes a first communication unit 141 connected to the data communication network 1 to enable data communication between a device connected to the data communication network 1 and the controller 200, and when the smart dispenser 12 supports another communication method that cannot be connected to the data communication network 1, a second communication unit 142 that performs mutual data communication according to the communication method of the smart dispenser 12. The communication method of the first communication unit 141 may be Internet Protocol (IP) communication, or the like, and the communication method of the second communication unit 142 may be RS-485, universal serial bus (USB), WiFi Direct, or the like.

The water treatment unit 150 is preferably configured when the drinking fountain 13 is configured in the potable water information terminal 11, and includes a water supply unit 151 that receives tap water from a connected water pipe and supplies to the water quality measuring unit 160 and the potable water release unit 170, and a water discharge unit 152 that discharges the water supplied to the water quality measuring unit 160 and the potable water release unit 170 to the outside.

The water quality measuring unit 160 measures the water quality of tap water supplied through the water treatment unit 150 and outputs water quality information for the measured water quality to the controller 200.

The water quality measuring unit 160 measures the turbidity, hydrogen ion concentration, residual chlorine, tap water temperature, and the like, of tap water, according to embodiments, and generates water quality state information including the measured turbidity, hydrogen ion concentration, residual chlorine concentration, temperature, and the like.

The potable water release unit 170 is preferably configured when the drinking fountain 13 is configured in the potable water information terminal 11, and when configured, it is controlled by the controller 200 and releases tap water supplied through the water treatment unit 150 to the outside through the drinking fountain 13.

The location measuring unit 180 receives location data from at least three Global Positioning System (GPS) satellites, calculates its own location (longitude and latitude), and outputs it to the controller 200.

The approach detection sensor unit 190 monitors whether there is an object approaching within a predetermined distance of the potable water information terminal 11, and outputs a detection signal to the controller 200 when an object is detected within the predetermined distance.

The controller 200 includes a terminal registration unit 210, a water quality information acquisition unit 230, a water quality information providing unit 240, a water quality management controller 250, and an information processing unit 260, and according to embodiments, further includes a potable water release controller 220, a promotional video output unit 270, and a carbon emission reduction amount calculating unit 280 to control the overall operation of the potable water information terminal 11 according to the present disclosure.

Specifically, the terminal registration unit 210 acquires the location information of the potable water information terminal 11 and stores it in the storage unit 110, and generates dispenser registration information including the location information and transmits it to the potable water living information providing unit 20 through the communication unit 140 to register the potable water information terminal and the location of the potable water information terminal with the potable water living information providing unit 20.

The location information may be the longitude and latitude where the potable water information terminal 11 is installed, or may be unique identification information of the potable water information terminal 11.

In the case that the location information is the longitude and latitude, it may be acquired by receiving input from a manager through a dispenser registration user interface means provided through the display unit 120 and the input unit 130 of the potable water information terminal 11, or may be a value measured through the location measuring unit 190.

In the case that the location information is the unique identification information, the potable water living information providing unit 20, which manages the potable water information terminal 11, must map and store installation location information (longitude and latitude) to the unique identification information of the registered potable water information terminal 11 and manage it.

The potable water release controller 220 may preferably be configured when the potable water release unit 170 and the drinking fountain 13 are configured.

When configured, the potable water release controller 220 receives a potable water release request command (hot water release, cold water release, lukewarm water release, etc.) through the display unit 120 and the input unit 130, supplies tap water supplied from the water treatment unit 150 as potable water to the potable water release unit 170 according to the inputted potable water release request command, performs water treatment according to the command in the potable water release unit 170 and then releases the tap water to the outside through the drinking fountain 13 so that the user can drink. The potable water release controller 220 releases tap water through the potable water release unit 170 and the drinking fountain 13 only when the release permission for tap water is set.

The potable water release controller 220 counts the number of potable water releases according to the potable water release request command and stores it in the storage unit 110, and measures the potable water release amount, calculates the cumulative release amount of the measured release amount, and stores it in the storage unit 110.

In addition, the potable water release controller 220 receives the number of releases, release amount, number of discharges, discharge amount, etc., from the smart dispenser 12 through the communication unit 140, and calculates the number of releases, total release amount, total number of discharges, total discharge amount, etc., within the water supply zone 2 and stores them in the storage unit 110.

When a water quality measuring event occurs, the water quality information acquisition unit 230 controls the water quality measuring unit 160 to measure water quality for measurement factors (turbidity, chlorine concentration, hydrogen ion concentration, water temperature, etc.) of stored tap water, and generates water quality state information including the measured water quality information and stores it in the storage unit 110. The water quality measuring event may occur when a potable water release request command is input through the potable water release controller 220, or may occur at regular intervals.

The water quality information providing unit 240 generates water quality measurement information including water quality state information, location information, and unique identification information of the potable water information terminal 11 acquired through the water quality information acquisition unit 230, transmits them to the potable water living information providing unit 20 and the smart dispenser 12 through the communication unit 140.

The water quality information providing unit 240 may transmit information such as the total number of discharges, discharge amount, total number of releases, total release amount, etc., along with the water quality measurement information to the potable water living information providing unit 20 and the smart dispenser.

In addition, according to an embodiment, the water quality information providing unit 240 may be configured to calculate the disinfection ability and food poisoning index of tap water based on the weather information acquired through the information processing unit 260 and the water quality state information acquired through the water quality information acquisition unit 230 and store them in the storage unit 110, and transmit them to the potable water living information providing unit 20 and the smart dispenser 12 through the communication unit 140. The information processing unit 260 may directly acquire the weather information from the potable water information providing unit 20 or the weather system 30. In addition, the potable water information terminal 11 and the smart dispenser 12 may further be provided with a temperature sensor (not shown), a humidity sensor (not shown), a rain sensor (not shown), etc., and in this case, the information processing unit 260 may acquire weather information including temperature, humidity, rain status, and the like, directly measured through the temperature sensor, humidity sensor, rain sensor, and the like. In this case, the water quality information providing unit 240 may calculate a unique food poisoning index for each of the potable water information terminal 11 and the smart dispenser 12.

The water quality information providing unit 240 may be configured to store information such as the water quality state information, number of discharges, discharge amount, number of releases, release amount, disinfection ability, food poisoning index, etc., and then display it on the display unit 120, or to display the information upon detection of an approach of a person through the approach detection sensor unit 190.

The water quality management controller 250 determines whether the water quality of the measurement factor is below a preset standard value and determines whether to discharge tap water and whether to permit release of tap water.

If the water quality exceeds the standard, the water quality management controller 250 determines the discharge of tap water and performs tap water discharge settings, and when provided with a dispenser 13, drives the water discharge unit 152 of the water treatment unit 150 to discharge tap water inside the potable water information terminal 11 to the outside.

If the water quality is below the standard value, the water quality management controller 250 determines the release of tap water and performs the potable water release permission setting.

In addition, when it is determined to discharge tap water, the water quality management controller 250 will transmit a tap water discharge command to the smart dispenser 12 in the same water supply zone 2. Then, each of all smart dispensers 12 will discharge the supplied tap water to the outside.

And when it is determined to release potable water, the water quality management controller 250 will transmit a potable water release permission command to the smart dispenser 12. Then, the smart dispenser 12 will set potable water release permission.

The potable water release controller 220 will be able to release tap water as potable water only when potable water release is permitted through the water quality management controller 220.

The information processing unit 260 receives potable water living information from the potable water living information providing unit 20 through the communication unit 140 and displays it on the display unit 120.

In the above description, a case where the potable water information terminal 11 calculates and provides the disinfection ability and food poisoning index of tap water is explained. However, according to another embodiment, the information processing unit 260 may be configured to receive the disinfection ability and food poisoning index of tap water from the potable water living information providing unit 20 through the communication unit 140 and display them on the display unit 120, and transmit them to the smart dispenser 12 to display them.

The information processing unit 260 extracts one or more of living information such as weather information, public information, convenience information, etc., and statistics information in which water quality state information, number of tap water discharges, discharge amount, number of tap water releases, release amount, etc., are staticized, from potable water living information to display the same in the preset living information display area of the display unit 120, and transmit the same to the smart dispenser 12 to display the same in the preset living information display area.

The information processing unit 260 extracts one or more of GIS-based potable water information in which water quality state information for each of the potable water information terminal 11 and nearby potable water information terminals 11 is mapped to GIS information, and GIS-based statistics information in which statistics information is mapped to GIS information from potable water living information and displays on the display unit 120, and displays geographical information, i.e., detailed water quality state information for a potable water information terminal selected from among potable water information terminals of map data, on a preset display area of the display unit 120 through the input unit 130.

In addition, the information processing unit 260 transmits one or more of the GIS-based potable water information and GIS-based statistics information to the smart dispenser 12 and displays in a preset display area. The information processing unit 260 may also be configured to display the potable water living information, GIS-based statistics information, etc., when a person's approach is detected through the approach detection sensor unit 190.

The carbon emission reduction amount calculating unit 280 defines a reduced carbon amount compared to using plastic bottled water according to a standard release amount (drinking amount) of tap water or a reduced carbon amount compared to using a water purifier according to the standard release amount of tap water, and calculates and stores a carbon emission reduction amount in correspondence to the defined carbon amount and the release amount of drinking tap water. The calculated carbon emission reduction amount is displayed on the display unit 120 and provided to the smart dispenser 12 and the potable water living information providing unit 20 through the communication unit 140.

Upon detection of a user approaching within a predetermined distance from the potable water information terminal 11 through the approach detection sensor unit 190, the promotional video output unit 270 loads a promotional video stored in the storage unit 110 or receives a promotional video from the potable water living information providing unit 20 through the communication unit 140 and displays on one or more of the display unit 120 and the smart dispenser 12. The promotional video may be a video to promote the method for use of the potable water information terminal 11 according to the present disclosure, the effect of using, and the cleanliness of tap water, and may be a video containing information on the carbon emission reduction amount (carbon reduction amount) according to the release amount of one or more of the release amount of tap water consumed through the corresponding potable water information terminal 11 and the release amount of tap water released through the potable water information terminal 11 by region, the release amount of tap water consumed through potable water information terminals 11 across the country.

FIG. 3 is a diagram illustrating a configuration of a smart dispenser of the system for providing safe water quality information of drinking water according to the present disclosure. Hereinafter, a description will be given with reference to FIG. 3, but a configuration that performs the same operation as the potable water information terminal 11 will be briefly described.

The smart dispenser 12 includes a dispenser storage unit 310, a dispenser input/output unit 320, a dispenser communication unit 330, a dispenser water treatment unit 340, a dispenser approach detection sensor unit 350, a dispenser potable water release unit 360 and a dispenser controller 370.

The dispenser storage unit 110 includes a program area for storing a control program for controlling the overall operation of the smart dispenser 12, a temporary area for temporarily storing data generated during execution of the control program, and a data area for semi-permanently storing data required for performing the control program and data generated during execution of the control program.

The dispenser input/output unit 320 may be configured to include input/output means corresponding to the display unit 120 and the input unit 130 in the same way as the potable water information terminal 11, and may be composed of only output means corresponding to the display unit 120 and may be configured to display only information.

The dispenser communication unit 330 may be configured to include one or more of a first communication means for performing data communication with the potable water information terminal 11 through the data communication network 1 and a second communication means directly connected to the potable water information terminal 11 to perform data communication.

The dispenser water treatment unit 340 includes a water supply unit 341 and a water discharge unit 342 and performs the same operations as the water treatment unit 150 of the potable water information terminal 11.

The dispenser approach detection sensor unit 350 also performs the same operation as the approach detection sensor unit 190 of the potable water information terminal 11.

The dispenser potable water release unit 360 also performs the same operation as the potable water release unit 170 of the potable water information terminal 11.

The dispenser controller 370 includes a potable water release controller 371 and an information processing unit 372, and further includes, when attempting to communicate with the potable water living information providing unit 20, a smart dispenser 12 registration unit (not shown) to control the overall operation of the smart dispenser 12. Registration of the smart dispenser 12 may be performed by the manager of the potable water living information providing unit 20.

The potable water release controller 371 is configured to, when a tap water discharge command is received from the potable water information terminal 11, discharge all the water supplied inside through the dispenser water treatment unit 340, and when a potable water release permission command is received, set potable water release permission.

The potable water release controller 371 controls the dispenser potable water release unit 360 according to the operation of the dispenser input/output unit 320 in a potable water release permission setting state and controls the potable water to be released to the outside.

The information processing unit 372 displays information received from the potable water information terminal 11 through the dispenser communication unit 330 on the display means of the dispenser input/output unit 320, and when the approach of an object is detected through the dispenser approach detection sensor unit 350, requests information to the potable water information terminal 11 through the dispenser communication unit 330 to display the received information on the display means of the dispenser input/output unit 320.

In addition, the information processing unit 372 acquires the number of releases and release amount released from the dispenser water treatment unit 340 through the dispenser potable water release unit 360 under the control of the potable water release controller 371, acquires the number of discharges and discharge amount discharged to the outside through the water discharge unit 342, and provides the acquired number of released, release amount, number of discharges, discharge amount to the potable water information terminal 11 through the dispenser communication unit 330.

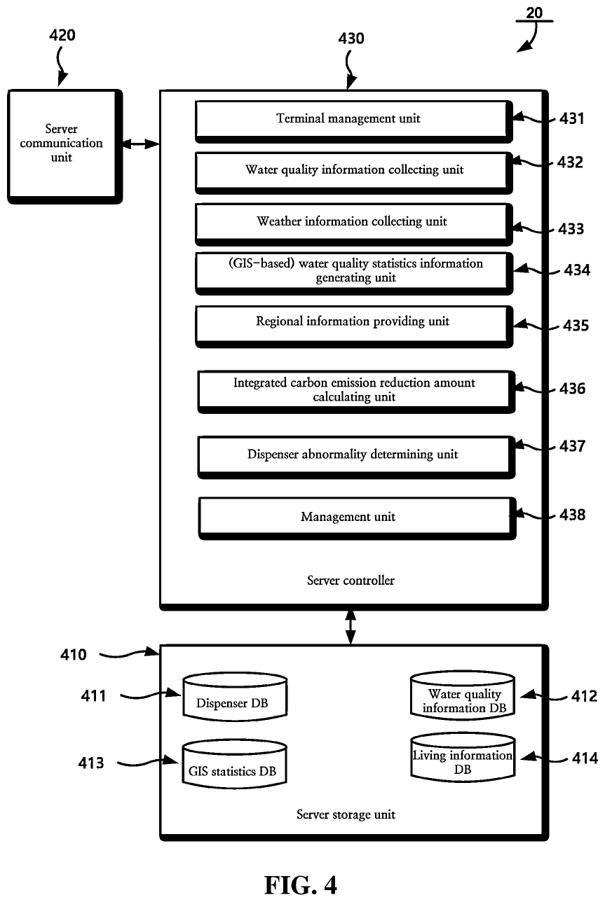

FIG. 4 is a diagram illustrating a configuration of a potable water living information providing unit of the system for providing safe water quality information of drinking water according to the present disclosure.

Referring to FIG. 4, the potable water living information providing unit 20 according to the present disclosure includes a server storage unit 410, a server communication unit 420, and a server controller 430.

The server storage unit 410 includes a program area for storing a control program for controlling the overall operation of the potable water living information providing unit 20, a temporary area for temporarily storing data generated during execution of the control program, and a data area for semi-permanently storing data required for performing the control program and data generated during execution of the control program. The data area includes a dispenser DB 411, a water quality information DB 412, a GIS statistics DB 413, a living information DB 414, etc., according to the present disclosure.

The dispenser DB 411 stores the dispenser registration information including the unique identification information and location information (longitude and latitude information) of the potable water information terminal 11, and according to embodiments, may further store smart dispenser registration information including unique identification information and location information (longitude and latitude information) of the smart dispenser 12.

The water quality information DB 412 stores the water quality measurement information for each potable water information terminal 11, and statistics information by region and time for the water quality measurement information, number of tap water discharges, discharge amount, number of tap water releases, release amount, and the like.

The GIS statistics DB 413 stores and is storing map data (geographic information), and stores GIS-based statistics information in which the statistics information is mapped to the location of the potable water information terminal 11 in the map data. It would be preferable that the map data maps water pipe information, water supply zone information for the water supply zone 2 where the same water quality is supplied, and the like.

The living information DB 414 stores regional weather information, regional public information, regional convenience information, and the like.

The server communication unit 420 connects to the data communication network 1 and allows devices connected to the data communication network 1 and the server controller 430 to perform mutual data communication.

The server controller 430 includes a terminal management unit 431, a water quality information collecting unit 432, a water quality statistics information generating unit 434, and a regional information providing unit 435, and according to embodiments, includes a weather information collecting unit 433, an integrated carbon emission reduction amount calculating unit 436, a dispenser abnormality determining unit 437, and a management unit 438 to control the overall operation of the potable water living information providing unit 20.

When dispenser registration information is received from any potable water information terminal 11 through the server communication unit 420, the terminal management unit 431 stores the dispenser registration information in the dispenser DB 411 to register the potable water information terminal 11. The terminal management unit 431 may receive unique identification information and location information of the potable water information terminal 11 from the manager and register the potable water information terminal 11. The terminal management unit 431 may also register the smart dispenser 12.

The water quality information collecting unit 432 collects water quality measurement information from the potable water information terminal 11 registered in the dispenser DB 411 through the server communication unit 420 and stores in the water quality information DB 412.

The weather information collecting unit 433 connects to the weather system 30 through the server communication unit 420 and acquires regional weather information from the weather system 30, or acquires weather information for each water supply zone 2 from the potable water information providing unit 10 and stores in the living information DB 414.

The water quality statistics information generating unit 434 generates statistics information by region, time, etc., for the water quality state information of the water quality measurement information for each potable water information terminal, the number of tap water discharges, and the number of potable water releases collected through the water quality information collecting unit 432 and stores in the water quality information DB 412, and then generates GIS-based statistics information in which GIS is combined with the water quality state information and statistics information in correspondence to the location of the potable water information terminal of the water quality measurement information and stores in the GIS statistics DB 413.

The regional information providing unit 435 generates water quality state information for each water supply zone 2 according to the location of the potable water information terminal 11 based on the water quality state information of the water quality measurement information acquired through the water quality information collecting unit 432, and transmits one or more of the water quality state information for each water supply zone 2, weather information collected through the weather information collecting unit 433, and GIS-based statistics information output from the water quality statistics information generating unit 434 to the potable water information terminal 11 corresponding to the region through the server communication unit 420.

According to another embodiment, the regional information providing unit 435 may calculate the disinfection ability and food poisoning index for each water supply zone 2 based on the water quality state information and weather information for each water supply zone 2 and provide to the potable water information terminal 11 in the corresponding water supply zone 2 as one of the potable water living information.

The integrated carbon emission reduction amount calculating unit 436 is configured according to the embodiment to calculate the carbon emission reduction amount for the release amount of tap water released from each potable water information providing unit 10 (or from each potable water information terminal 11) collected, and the carbon emission reduction amount for a certain area (large apartment complex, etc.) or a certain legal area (legal district, legal district, legal city, etc.) including a plurality of water supply zones 2 and store the same in the server storage unit 410.

The carbon emission reduction amount may be mapped to GIS by the water quality statistics information generating unit 434 and stored as included in the GIS-based statistics information.

When water quality of any potable water information terminal 11 falls below a predetermined standard value compared to water quality of other adjacent potable water information terminals 11 based on the water quality measurement information collected through the water quality information collecting unit 432, the dispenser abnormality determining unit 437 determines that there is an abnormality in the potable water information terminal 11 of poor water quality or that there is an abnormality in the water pipe supplying tap water to the potable water information terminal 11, and transmits potable water abnormality notification information to a preset manager terminal 40. It would be preferable for the potable water information terminal 11 and other adjacent potable water information terminals 11 to be located in a water pipe with the same water quality.

The management unit 438 provides a living information input means for inputting public interest information, convenience information, etc., into the manager terminal 40, classifies public interest information and convenience information input through the living information input means by region and stores in the living information DB 414.

The stored public interest information, convenience information, etc., will be provided to the corresponding potable water information terminal 11 through the regional information providing unit 435.

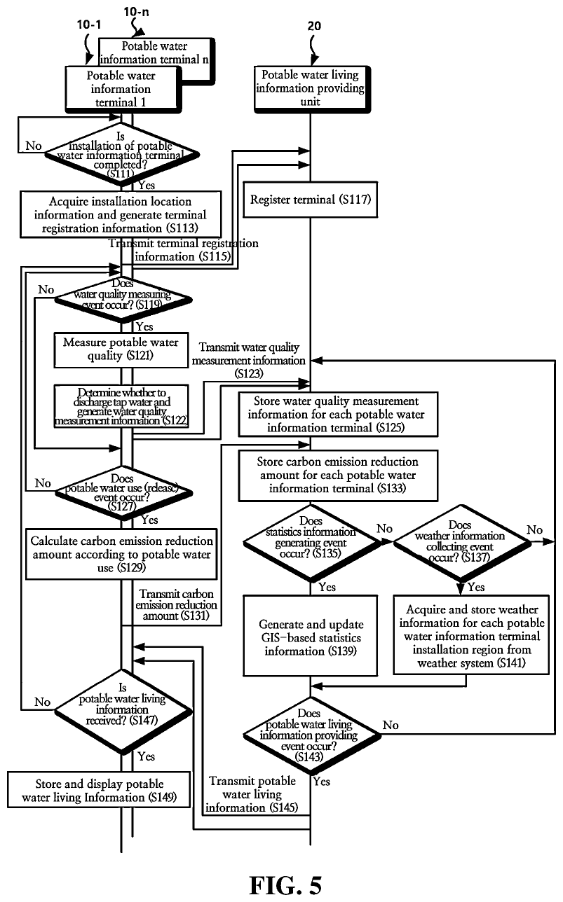

FIG. 5 is a flowchart illustrating a method of supplying safe potable water using the system for providing safe water quality information of drinking water according to an embodiment of the present disclosure.

Referring to FIG. 5, the potable water information terminal 11 is connected to a water pipe when initially driven and monitors whether installation of the potable water information terminal is completed so that tap water flows in (S111).

When installation is complete, the potable water information terminal 11 acquires installation location information through a location registration user interface means through the display unit 120 and the input unit 130 or through the location measuring unit 190, and generates dispenser registration information including the acquired location information (S113).

When the dispenser registration information is generated, the potable water information terminal 11 transmits the dispenser registration information to the potable water living information providing unit 20 and requests registration of the potable water information terminal 11 (S115), and receives the registration completion information from the potable water living information providing unit 20 (not shown) to complete the registration.

When the dispenser registration information is received from the potable water information terminal 11, the potable water living information providing unit 20 stores it in the dispenser DB 411 of the server storage unit 410 and registers the potable water information terminal 11 (S117).

The registered potable water information terminal 11 monitors whether a water quality measuring event occurs (S119), and when a water quality measuring event occurs, measures the water quality of the tap water stored in the potable water information terminal 11 through the water quality measuring unit 160 (S121).

When the water quality is measured, the potable water information terminal 11 determines whether tap water should be discharged because the measured water quality exceeds a preset standard value to determine whether to discharge tap water, and generates water quality measurement information including the water quality state information, number of tap water discharges, tap water discharge amount according to whether tap water is discharged, number of potable water releases, potable water release amount, disinfection ability, food poisoning index, etc., previously stored (S122). Upon discharge determination of tap water, the potable water information terminal 11 will control the water discharge unit 152 of the water treatment unit 150 to discharge all tap water supplied to the potable water information terminal 11 to the outside.

When water quality measurement information is generated, the potable water information terminal 11 transmits the water quality measurement information to the potable water living information providing unit 20 and the smart dispenser 12 (S123).

When water quality measurement information is received from the potable water information terminal 11, the potable water living information providing unit 20 stores the water quality measurement information for each potable water information terminal in the water quality information DB 412 of the server storage unit 410 (S125).

When transmitting water quality measurement information or a water quality measuring event does not occur, the potable water information terminal 11 monitors whether a potable water release (drinking) event occurs (S127). The potable water release event may occur through the display unit 120 and input unit 130 of the potable water information terminal 11.

When a potable water release event occurs and when water release is permitted because the water quality measurement information is below the standard value, the potable water information terminal 11 releases tap water as potable water through the potable water release unit 170 and calculate the carbon emission reduction amount according to the potable water release amount (S129).

When the carbon emission reduction amount is calculated, the potable water information terminal 11 will transmit only the calculated carbon emission reduction amount as water quality measurement information to the potable water living information providing unit 20 and the smart dispenser 12, or transmit water quality measurement information including previously measured water quality state information, carbon emission reduction amount, number of tap water discharges, tap water discharge capacity, number of potable water releases, potable water release amount, etc., to the potable water living information providing unit 20 and the smart dispenser 12 (S131).

The potable water living information providing unit 20, which has received the water quality measurement information including the carbon emission reduction amount, will map and store it to the corresponding potable water information terminal 11 of the water quality information DB 412 of the storage unit 410 (S133).

When water quality measurement information begins to be received from the potable water information terminals 11 through the above-described process, the potable water living information providing unit 20 monitors whether a statistics information generating event occurs (S135) or a weather information collecting event occurs (S137). The statistics information generating event and weather information collecting event may occur automatically at a predetermined period, may be generated by a manager, or may occur when water quality measurement information is received in a predetermined time unit from all of the installed potable water information terminals 11.

When a statistics information generating event occurs, the potable water living information providing unit 20 generates statistics information, then generates GIS-based statistics information and stores or updates in the GIS statistics DB 413 of the server storage unit 410 (S139).

When a weather information collecting event occurs, the potable water living information providing unit 20 connects to the weather system 30, collects weather information for each potable water information terminal installation region, and stores in the living information DB 414 of the server storage unit 410 (S141).

When the above information begins to be generated, the potable water living information providing unit 20 monitors whether a potable water living information providing event occurs (S143), and when a potable water living information providing event occurs, transmits each regional potable water living information to the potable water information terminal 11 of the corresponding region (S145).

The potable water information terminal 11 monitors whether potable water living information is received from the potable water living information providing unit 20 while performing steps S119 to S131 (S147), and upon receiving the potable water living information, displays the potable water living information on the display unit 120 (S149).

In addition, according to another embodiment, the potable water information terminal 11 may be configured to calculate the disinfection ability and food poisoning index for the tap water of the water supply zone 2 based on the measured weather information of temperature and humidity and water quality measurement information of the corresponding water supply zone 2, and transmit the calculated disinfection ability and food poisoning index to the potable water living information providing unit 20 and the smart dispenser 12. The temperature and humidity may be information acquired from the weather system 30, or may be information measured through a temperature sensor and a humidity sensor configured in the potable water information terminal 11 itself.

In addition, according to another embodiment, the potable water living information providing unit 20 may be configured to calculate the disinfection ability and food poisoning index for each water supply zone 2, each area unit, and each legal area based on the collected information, transmit the disinfection ability and food poisoning index of the corresponding water supply zone to the potable water information terminal 11 of the corresponding water supply zone 2, and optionally provide the disinfection ability and food poisoning index of tap water for each area unit, each legal area, and the adjacent water supply zone 2 to the potable water information terminal 11.

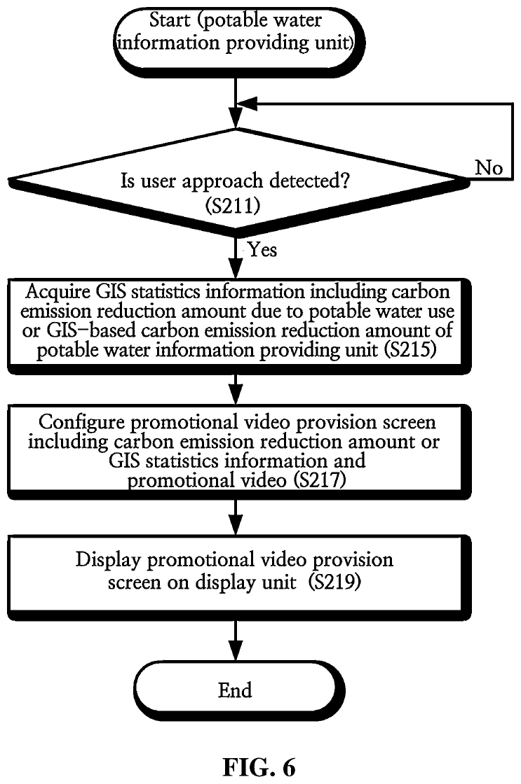

FIG. 6 is a flowchart illustrating a promotional video providing method of the potable water information terminal of the method of supplying safe potable water using the system for providing safe water quality information of drinking water according to an embodiment of the present disclosure.

Referring to FIG. 6, the potable water information terminal 11 monitors whether a user entering within a predetermined distance from the potable water information terminal 11 is detected through the approach detection sensor unit 195 (S211).

When a user entering within the predetermined distance is detected, the potable water information terminal 11 acquires the measured and stored water quality state information, carbon emission reduction amount due to use of tap water provided as potable water by itself, disinfection ability, food poisoning index, etc., from the storage unit 110, or acquires GIS-based statistics information including GIS-based water quality state information, carbon emission reduction amount information, disinfection ability, food poisoning index, etc., from the potable water living information providing unit 20 (S215).

When information such as the water quality state information, carbon emission reduction amount, disinfection ability, food poisoning index, or GIS-based statistics information is acquired, the potable water information terminal 11 configures a promotional video provision screen by combining one or more of the water quality state information, carbon emission reduction amount, disinfection ability, food poisoning index, and GIS-based statistics information with the promotional video or promotional graphic stored in the storage unit 110 or acquired from the potable water living information providing unit 20 (S217), and then displays on the display unit 120 and transmits to the smart dispenser 12 for display (S219).

Those skilled in the art will easily understand that the present disclosure is not limited to the above-described typical preferred embodiments, but can be implemented by various improvements, changes, substitutions, or additions without departing from the gist of the present disclosure. If the implementation by such improvement, change, substitution or addition falls within the scope of the appended claims below, the technical idea thereof should also be regarded as belonging to the present disclosure.

### EXPLANATION OF SYMBOLS

1: Data communication network
2: Water supply zone
10: Potable water information providing unit
11: Potable water information terminal
12: Smart dispenser
13: Drinking fountain
14: Information receiving unit
20: Potable water living information providing unit
30: Weather system
40: Manager terminal unit
110: Storage unit
120: Display unit
130: Input unit
140: Communication unit
150: Water treatment unit
151: Water supply unit
152: Water discharge unit
160: Water quality measuring unit
170: Potable water releasing unit
180: Location measuring unit
190: Approach detection sensor unit
200: Controller
210: Terminal registration unit
220: Potable water release controller
230: Water quality information acquisition unit
240: Water quality information providing unit
250: Water quality management controller
260: Information processing unit
270: Promotional video output unit
280: Carbon emission reduction amount calculating unit
310: Dispenser storage unit
320: Dispenser input/output unit
330: Dispenser communication unit
340: Dispenser water treatment unit
350: Dispenser approach detection sensor unit
360: Dispenser potable water releasing unit
370: Dispenser controller
371: Potable water release controller
372: Information processing unit
410: Server storage unit
411: Dispenser DB
412: Water quality information DB
413: GIS statistics DB
414: Living information DB
420: Server communication unit
430: Server controller
431: Terminal management unit
432: Water quality information collecting unit
433: Weather information collecting unit
434: Water quality statistics information generating unit
435: Regional information providing unit
436: Integrated carbon emission reduction amount calculating unit
437: Dispenser abnormality determining unit
438: Management unit

What is claimed is:

1. A system for providing safe water quality information of drinking water, the system comprising:
a plurality of potable water information providing units installed in different water supply zones, each configured to receive tap water from a water pipe of a water supply zone of the same water quality, measure water quality of the tap water, if the measured water quality exceeds a standard value, perform a discharge setting that commands to discharge the supplied tap water, and if the measured water quality is below the standard value, perform a permission setting that permits to supply as potable water which is drinking water, display and then transmit water quality measurement information comprising water quality state information according to the measured water quality and own location information, display environmental improvement information due to use of the tap water as drinking water and potable water reliability information based on the water quality state information, and display received potable water living information; and
a potable water living information providing unit configured to receive the water quality measurement information from the potable water information providing unit, analyze water quality state based on the location information of the water quality measurement information to generate regional potable water information, and generate the potable water living information comprising the regional potable water information to transmit it to the potable water information providing unit.

2. The system of claim 1, wherein the potable water information providing unit comprises:
a potable water information terminal installed at a random location of the water supply zone and configured to receive tap water from the water pipe of the water supply zone and measure water quality of the tap water, display and then transmit the water quality measurement information comprising the water quality state information for the measured water quality, the environmental improvement information, and the potable water reliability information based on the water quality state information, receive, display, and then transmit the potable water living information, if the water quality exceeds the standard value, perform the discharge setting to transmit a tap water discharge command, and if the water quality is below the standard value, perform the permission setting to permit the tap water as potable water which is drinking water, and transmit a potable water release permission command; and
a smart dispenser installed at a random location of the water supply zone and configured to receive tap water from the water pipe of the water supply zone, discharge the supplied tap water to outside when the tap water discharge command is received from the potable water information terminal, set a potable water release permission when the potable water release permission command is received and release the tap water upon tap water release request while the potable water release permission is set, and receive and display the water quality state information, the environmental improvement information, the potable water reliability information, and the potable water living information from the potable water information terminal.

3. The system of claim 2, wherein the potable water information terminal comprises:
a storage unit configured to store own location information;
a communication unit configured to perform data communication with the potable water living information providing unit and the smart dispenser;
a display unit;
a water quality measuring unit configured to receive the tap water to measure and output the water quality of the tap water; and
a controller configured to measure the water quality of the tap water through the water quality measuring unit, display and then transmit the water quality measurement information comprising the water quality state information for the measured water quality, the environmental improvement information, and the potable water reliability information based on the water quality state information, receive, display and then transmit the potable water living information, if the water quality exceeds the standard value, perform the discharge setting and transmit the tap water discharge command to the smart dispenser through the communication unit, and if the water quality is below the standard value, perform the permission setting to permit the tap water as drinking water for drinking and transmit the potable water release permission command to the smart dispenser through the communication unit, and further include the number of discharges, a discharge amount, the number of releases, and a release amount of tap water according to discharge and release of tap water through the smart dispenser in the water quality measurement information and transmit the water quality measurement information to a potable water living information management unit through the communication unit.

4. The system of claim 3, wherein the potable water information terminal further comprises an input unit configured to, together with the display unit, provide a user interface means through which a potable water information terminal user inputs and selects information for using the potable water information terminal, and
wherein the controller comprises:
a terminal registration unit configured to acquire the location information of the potable water information terminal and store the location information in the storage unit, generate dispenser registration information comprising the location information and transmit the dispenser registration information to the potable water living information providing unit through the communication unit to register the potable water information terminal and a location of the potable water information terminal;
a water quality information acquisition unit configured to, when a water quality measuring event occurs, acquire water quality information of the tap water through the water quality measuring unit, and generate and output the water quality state information comprising the acquired water quality information;
a water quality management controller configured to acquire the water quality state information from the water quality information acquisition unit, determine whether the water quality of the water quality state information is below a preset standard value to determine whether to discharge tap water and whether to permit release, and when it is determined to discharge tap water, perform the discharge setting and transmit the tap water discharge command to the smart dispenser, and when it is determined to permit release, set the potable water release permission and transmit the potable water release permission command to the smart dispenser;
a carbon emission reduction amount calculating unit configured to define a reduced carbon amount compared to using plastic bottled water according to a standard release amount (drinking amount) of tap water or a reduced carbon amount compared to using a water purifier according to the standard release amount of tap water, and calculate and store a carbon emission reduction amount which is the environmental improvement information, in correspondence to the defined carbon amount and the release amount of drinking tap water;
a water quality information providing unit configured to acquire the water quality state information and the carbon emission reduction amount, display the water quality measurement information comprising the water quality state information and the carbon emission reduction amount on the display unit, and transmit them to the potable water living information providing unit and the smart dispenser through the communication unit; and
an information processing unit configured to receive the potable water living information from the potable water living information providing unit through the communication unit and display it on the display unit.

5. The system of claim 4, wherein the potable water living information comprises GIS-based potable water information in which water quality state information for each of the potable water information terminal and nearby potable water information terminals is mapped to GIS information, and
wherein the information processing unit is configured to display the GIS-based potable water information on the display unit, and
display detailed water quality state information for the potable water information terminal selected through the input unit on the display unit.

6. The system of claim 5, wherein the water quality information providing unit is configured to count the number of potable water releases through a potable water release controller and the smart dispenser and the number of tap water discharges through the water quality management controller and the smart dispenser, and include the number of potable water releases, the release amount according to the number of potable water releases, the number of tap water discharges, and the discharge amount according to the number of discharges in the water quality measurement information and transmit the water quality measurement information to the potable water living information providing unit, and
the information processing unit is configured to extract, from the potable water living information comprising statistics information generated by the potable water living information providing unit by combining with GIS after generating statistics based on the water quality state information of the water quality measurement information, the number of potable water discharges and discharge amount, and the number of potable water releases and release amount received from the potable water information terminal, the statistics information and display the statistics information on the display unit.

7. The system of claim 2, wherein the potable water living information providing unit comprises:
a server storage unit comprising a water quality information DB storing water quality state information over time for each of potable water information terminals;
a server communication unit configured to perform mutual data communication with the potable water information terminals; and
a server controller configured to receive the water quality measurement information from the potable water information terminal through the server communication unit, store the water quality measurement information in the server storage unit, analyze the water quality state based on the location information of the water quality measurement information to generate the regional potable water information, and generate the potable water living information comprising the regional potable water information to transmit it to the potable water information terminal.

8. The system of claim 7, wherein the server storage unit further comprises a dispenser DB storing dispenser registration information comprising potable water information terminal installation location information for each potable water information terminal, and
wherein the server controller comprises:
a terminal management unit configured to, when the dispenser registration information is received from any potable water information terminal through the server communication unit, store and register the dispenser registration information in the dispenser DB;
a water quality information collecting unit configured to collect and output the water quality measurement information from the potable water information terminal registered in the dispenser DB through the server communication unit;
a water quality statistics information generating unit configured to, after generating statistics information for the water quality state information of the water quality measurement information for each potable water information terminal, the number of discharges and a discharge amount of tap water, and the number of releases and a release amount of potable water collected through the water quality information collecting unit, generate and output GIS-based statistics information in which GIS is combined with the water quality state information and the statistics information in correspondence to the location of the potable water information terminal of the water quality measurement information; and
a regional information providing unit configured to generate regional water quality state information according to the location of the potable water information terminal based on the water quality state information of the water quality measurement information acquired through the water quality information collecting unit, and transmit one or more of the regional water quality state information and the GIS-based statistics information output from the water quality statistics information generating unit to the potable water information terminal corresponding to the region through the server communication unit.

9. The system of claim 8, wherein the server controller further comprises a dispenser abnormality determining unit configured to, when water quality of any potable water information terminal falls below a predetermined standard value compared to water quality of other adjacent potable water information terminals based on the water quality measurement information collected through the water quality information collecting unit, determine that there is an abnormality in the potable water information terminal of poor water quality or that there is an abnormality in the water pipe supplying tap water to the potable water information terminal, and transmit potable water abnormality notification information to a preset manager terminal.

10. The system of claim 8, wherein the server controller further comprises a weather information collecting unit configured to collect and output weather information for each water supply zone from any one of a weather system and the potable water information terminal of the potable water information providing unit for the water supply zone through the server communication unit, and
the regional information providing unit is configured to, based on the weather information for each water supply zone and the water quality state information for each water supply zone, calculate in real time a disinfection ability and a food poisoning index for each water supply zone, which are the potable water reliability information, and further provide them to the potable water information terminal of the corresponding potable water information providing unit for each water supply zone.

11. A method of supplying safe potable water using a system for providing safe water quality information of drinking water, the method comprising:
a potable water information providing unit registrating process in which a potable water information providing unit installed in a water supply zone where tap water of the same water quality is supplied acquires own location information and stores it in a storage unit, and generates dispenser registration information comprising the location information and transmits it to a potable water living information providing unit to register own location;
a water quality measurement information providing process in which the potable water information providing unit receives tap water from a water pipe of the water supply zone, measures water quality of the tap water, if the measured water quality exceeds a standard value, performs a discharge setting, and if the measured water quality is below the standard value, performs a permission setting to permit as potable water which is drinking water, and transmits water quality measurement information comprising water quality state information according to the measured water quality and own location information;
a potable water living information providing process in which the potable water living information providing unit receives the water quality measurement information from the potable water information providing unit, analyzes water quality state based on the location information of the water quality measurement information to generate regional potable water information comprising regional water quality state information, and generates potable water living information comprising the regional potable water information to transmit the potable water living information to the potable water information providing unit; and
a potable water information display process in which the potable water information providing unit receives and display the received potable water living information, and generates and displays environmental improvement information due to use of tap water as drinking water based on the water quality measurement information and the potable water living information and potable water reliability information based on the water quality state information.

12. The method of claim 11, wherein the water quality measurement information providing process comprises:
a water quality management controlling step in which a potable water information terminal of the potable water information providing unit receives tap water from the water pipe of the water supply zone and measures water quality of the tap water, determine whether the water quality exceeds or is below the standard value, if the water quality exceeds the standard value, performs the discharge setting by determining to discharge the supplied tap water and notifies the discharge setting of the tap water by transmitting a tap water discharge command to one or more smart dispensers of the water supply zone, and if the water quality is below the standard value, performs the permission setting to permit the tap water as potable water which is drinking water, and sets a drinking permission by transmitting a potable water release permission command to the smart dispenser; and
a tap water treating step in which each smart dispenser of the potable water information providing unit discharges the supplied tap water upon the discharge setting of the tap water, and when a release request of the tap water occurs upon the drinking permission setting of the tap water, releases the tap water as drinking water.

13. The method of claim 12, wherein the potable water information display process comprises:
a water quality information providing step in which a controller of the potable water information terminal acquires the water quality state information from a water quality information acquisition unit through a water quality information providing unit, displays water quality measurement information comprising the water quality state information on a display unit, and transmits the water quality measurement information to the potable water living information providing unit and the smart dispenser through a communication unit; and
an information processing step in which the controller displays the environmental improvement information due to use of tap water as drinking water and the potable water reliability information based on the water quality state information through the water quality information acquisition unit, receives the potable water living information from the potable water living information providing unit through the information processing unit and displays it on the display unit.

14. The method of claim 13, wherein the potable water information display process further comprises a location information acquiring step in which the controller receives location data from a plurality of Global Positioning System (GPS) satellites through a location measuring unit and measures own location, and outputs measured location information to the controller, and
wherein the controller acquires, in a dispenser registration step, the location information through the location measuring unit.

15. The method of claim 13, wherein the potable water information display process further comprises:
a user approach detecting step in which the controller detects users approaching within a predetermined distance through an approach detection sensor unit and outputs a detection signal; and
a promotional video output step in which, when the detection signal is input from the approach detection sensor unit, the controller acquires a promotional video promoting features and effects of the potable water information terminal through a promotional video output unit and displays the promotional video on the display unit.

16. The method of claim 13, wherein the potable water living information comprises GIS-based potable water information in which water quality state information for each of the potable water information terminal and nearby potable water information terminals is mapped to GIS information, and
wherein, in the information processing step, the information processing unit displays the GIS-based potable water information on the display unit, and displays detailed water quality state information for the potable water information terminal selected through an input unit on the display unit.

17. The method of claim 16, wherein, in the water quality information providing step, the controller counts the number of potable water releases through a potable water release controller and the number of tap water discharges through a water quality management controller, and includes the number of potable water releases, a release amount according to the number of potable water releases, the number of tap water discharges, and a discharge amount according to the number of tap water discharges in the water quality measurement information and transmits the water quality measurement information to the potable water living information providing unit, and
in the information processing step, the controller extracts, from the potable water living information comprising statistics information generated by the potable water living information providing unit by combining with GIS after generating statistics based on the water quality state information of the water quality measurement information, the number of potable water discharges and discharge amount, the number of potable water releases and release amount received from the potable water information terminal, the statistics information and displays the statistics information on the display unit.

18. The method of claim 12, wherein the potable water living information providing process comprises:
a water quality information collecting step in which a server controller of the potable water living information providing unit collects the water quality measurement information from a potable water information terminal registered in a dispenser DB through a water quality information collecting unit;
a water quality statistics information generating step in which, after generating statistics information for the water quality state information of the water quality measurement information for each potable water information terminal, the number of discharges and a discharge amount of tap water, and the number of releases and a release amount of potable water collected from the water quality information collecting unit, the server controller generates and outputs GIS-based statistics information in which GIS is combined with the water quality state information and the statistics information in correspondence to the location of the potable water information terminal of the water quality measurement information through a water quality statistics information generating unit; and
a regional information providing step in which the server controller generates regional water quality state information according to the location of the potable water information terminal based on the water quality state information of the water quality measurement information input from the water quality information collecting unit, and transmits one or more of the regional water quality state information and the GIS-based statistics information output from the water quality statistics information generating unit to the potable water information terminal corresponding to the region through the server communication unit through a regional information providing unit.

19. The method of claim 18, wherein the potable water living information providing process further comprises a weather information collecting step in which the server controller collects and outputs weather information for each water supply zone from any one of a weather system and the potable water information providing unit through a weather information collecting unit, and
wherein, in the regional information providing step, the server controller calculates in real time, based on the weather information for each water supply zone and the water quality state information for each water supply zone, a disinfection ability and a food poisoning index for each water supply zone, which are the potable water reliability information, and further provides them to the potable water information terminal of the corresponding potable water information providing unit for each water supply zone through the regional information providing unit.

20. The method of claim 18, wherein the potable water living information providing process further comprises a dispenser abnormality determining step in which, when water quality of any potable water information terminal falls below a predetermined standard value compared to water quality of adjacent potable water information terminals based on the water quality measurement information collected from the water quality information collecting unit, the server controller determines that there is an abnormality in the potable water information terminal of poor water quality or that there is an abnormality in a water pipe supplying tap water to the potable water information terminal, and transmits potable water abnormality notification information to a preset manager terminal through a dispenser abnormality determining unit.
